(19) United States
(12) Patent Application Publication
(10) Pub. No.: US 2007/0266542 A1
(43) Pub. Date: Nov. 22, 2007

(54) Radiopaque marker for intraluminal medical device

(75) Inventor: Jeffry Scott Melsheimer, Springville, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 11/800,218

(22) Filed: May 4, 2007

Related U.S. Application Data
(60) Provisional application No. 60/798,880, filed on May 8, 2006.

(51) Int. Cl. A61F 2/82; A61F 2/06; B23P 11/00

(52) U.S. Cl. 29/522.1; 623/1.34; 623/901; 29/428; 29/505

(57) ABSTRACT

An intraluminal medical device including at least one radiopaque marker is described. The radiopaque marker includes an eyelet having a thickness and an opening extending through the thickness from a first side to a second side. The opening is defined by at least an inclined surface generally facing toward the first side and a recessed region generally facing toward the second side. The recessed region is disposed about only a portion of a perimeter of the opening. A radiopaque rivet is disposed within the opening. The radiopaque rivet includes a first portion engaging the inclined surface and a second portion engaging the recessed region.

### RELATED APPLICATIONS

The present patent document claims the benefit of the filing date under 35 U.S.C. §119(e) of Provisional U.S. Patent Application Ser. No. 60/798,880, filed on May 8, 2006, which is hereby incorporated by reference.

### TECHNICAL FIELD

The present disclosure relates to intraluminal medical devices, in particular to intraluminal medical devices having radiopaque markers.

### BACKGROUND

A stent is a tubular support structure that may be implanted within a body vessel to treat blockages, occlusions, narrowing ailments and other related problems that restrict flow through the vessel. When delivered to the site of a constricted vessel and expanded from a compressed diameter to an expanded diameter, the stent exerts a radial force on the vessel wall and prevents it from closing.

In order to effectively treat blockages, occlusions and other ailments that restrict flow through a body vessel, it is important that the stent be precisely placed at the site of the constriction. One approach to achieve precise stent placement is to attach radiopaque markers to the stent to permit visualization of the stent from outside the body using x-ray fluoroscopy. During the implantation procedure, the position of the markers—and thus the position of the stent—may be monitored using a fluoroscope. The x-ray visibility of stents made of metals such as nickel and titanium may be substantially improved by using markers formed from heavier metals such as platinum or gold, which produce higher x-ray contrast. Traditionally, such radiopaque markers have taken the form of a rivet or pin which is inserted through an opening in the stent wall and held in place by a head formed on both ends of the marker.

Despite their usefulness in improving the visibility of stents during insertion into a body vessel, radiopaque markers are not without shortcomings. One problem is that the protruding heads that traditionally have secured markers in place may also significantly increase the effective wall thickness of the stent. This can be problematic due to space constraints within the delivery system used to transport and deploy a stent within a vessel. In the case of a self-expandable stent, the delivery system may include an inner catheter or member having one or more lumens, a retaining sheath to keep the stent in a compressed configuration during delivery, and the stent itself. It is desirable to make the delivery system as compact as possible for transport through the vessel. In some cases, the amount of protrusion of a head of a radiopaque marker may be as large as the wall thickness of the stent. Besides increasing the profile of the medical device and the delivery system, a protruding head may hamper deployment efforts by interfering with the removal of the retaining sheath. Once the stent is positioned within the vessel adjacent to the site to be treated, the retaining sheath must be retracted to allow the stent to expand to support the vessel wall.

To get around these shortcomings, there have been attempts to eliminate the protruding heads of traditional marker designs and to use other approaches for securing the radiopaque marker to the stent. However, in alternative designs the marker may be easily dislodged or may lack structural integrity.

### SUMMARY

An intraluminal medical device including at least one radiopaque marker is described. Preferably, the marker is at least partially flush with the surface of the medical device and advantageously does not substantially increase the profile of the medical device for delivery into a body vessel. Also, the marker engages and does not easily dislodg from the medical device. It is also preferred that the device be manufactured by simple manufacturing processes.

According to one embodiment, the radiopaque marker includes an eyelet having a thickness and an opening extending through the thickness from a first side to a second side. The opening is defined by at least an inclined surface generally facing toward the first side and a recessed region generally facing toward the second side. The recessed region is disposed about only a portion of a perimeter of the opening. A radiopaque rivet is disposed within the opening. The radiopaque rivet includes a first portion engaging the inclined surface and a second portion engaging the recessed region.

According to another embodiment, the radiopaque marker includes an eyelet having a thickness and an opening extending through the thickness from a first side to a second side. The opening is defined by at least an inclined surface generally facing toward the first side and two recessed regions positioned opposite of each other about a perimeter of the opening and generally facing toward the second side. Each recessed region is disposed about only a portion of a perimeter of the opening. A radiopaque rivet is disposed within the opening. The radiopaque rivet includes a first portion engaging the inclined surface and a second portion engaging the recessed regions. The eyelet also includes an exterior surface having a first radius of curvature and an interior surface having a second radius of curvature. The exterior surface is substantially flush with an exterior surface of the radiopaque rivet and the interior surface is substantially flush with an interior surface of the radiopaque rivet.

Also described is a method of making an intraluminal medical device including at least one radiopaque marker. A thin-walled tube is provided, and a generally tubular structure and an eyelet are formed from the thin-walled tube. The forming of the eyelet includes forming an opening extending through a thickness of the eyelet from a first end to a second end. The forming of the opening includes at least forming an inclined surface generally facing toward the first end and forming a recessed region generally facing toward the second end of the eyelet. A radiopaque rivet is inserted into the opening of the eyelet and secured such that a first portion of the radiopaque rivet engages the inclined surface and a second portion of the radiopaque rivet engages the recessed region.

### BRIEF DESCRIPTION OF THE DRAWINGS

FIG. 1 shows a portion of an exemplary intraluminal medical device with radiopaque markers at one end;

FIG. 2 shows the exterior surface of a radiopaque marker according to one embodiment;

FIG. 3 shows the interior surface of the radiopaque marker according to one embodiment;

FIGS. 4A-4D show different views of a radiopaque rivet and eyelet according to one embodiment prior to assembly and forming into a radiopaque marker, where FIG. 4A is an exploded plan view; FIG. 4B is an exploded sectional view along section 4B-4B of the eyelet in FIG. 4A; FIG. 4C is an exploded sectional view along section 4C-4C of the eyelet in FIG. 4A; and FIG. 4D is an exploded perspective view;

FIGS. 5A-5D show different views of a radiopaque rivet and eyelet according to another embodiment prior to assembly and forming into a radiopaque marker, where FIG. 5A is an exploded plan view; FIG. 5B is an exploded sectional view along section 5B-5B of the eyelet in FIG. 5A; FIG. 5C is an exploded sectional view along section 5C-5C of the eyelet in FIG. 5A; and FIG. 5D is an exploded perspective view;

FIGS. 6A-6C show a process of forming two recessed regions in the eyelet according to one embodiment;

FIGS. 6D-6F show alternative embodiments of the shape of the recessed regions formed in the eyelet;

FIGS. 7A-7B show a manufacturing process of a radiopaque marker according to one embodiment, including inserting the radiopaque rivet into the eyelet and securing the radiopaque rivet within the eyelet;

FIGS. 8A-8B show a manufacturing process of a radiopaque marker according to another embodiment, including inserting the radiopaque rivet into the eyelet and securing the radiopaque rivet within the eyelet; and

FIG. 9 is a sectional view of a radiopaque marker according to one embodiment after insertion and securing of the radiopaque rivet within the eyelet.

### DETAILED DESCRIPTION

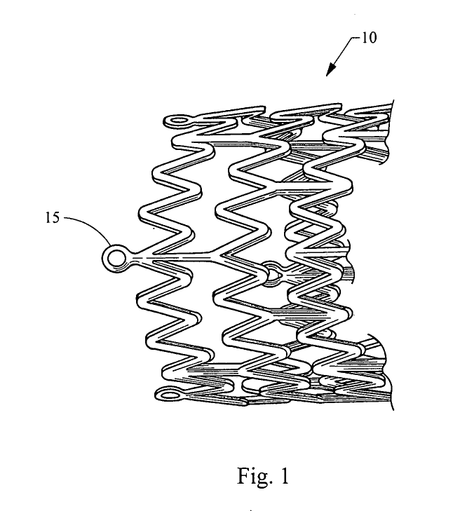

Shown in FIG. 1 is an intraluminal medical device including at least one radiopaque marker 15 disposed at an end of a generally tubular structure 10. According to this embodiment, the radiopaque markers 15 extend along a direction parallel to the longitudinal axis of the tubular structure 10. The medical device may be, for example, a stent, as shown, or a stent graft, vascular graft, filter, embolization coil, catheter, guide wire or other intraluminal medical device.

Preferably, at least one marker 15 is disposed at each end of the tubular structure 10. In some embodiments, two, three, four, five, or six radiopaque markers 15 may be disposed at each end of the tubular structure 10. Typically, the marker 15 extends only a short distance (e.g., less than about 1 mm) beyond the end of the tubular structure 10. Thus, the length and precise position of the medical device within a body vessel may be accurately determined using an x-ray imaging device, such as a fluoroscope. If desired, radiopaque markers may also be disposed between the ends of the tubular structure.

In embodiments in which the tubular structure 10 includes more than one marker 15, the markers 15 may be symmetrically disposed about the circumference. Asymmetric arrangements of markers 15 about the circumference of the tubular structure 10 may also be used.

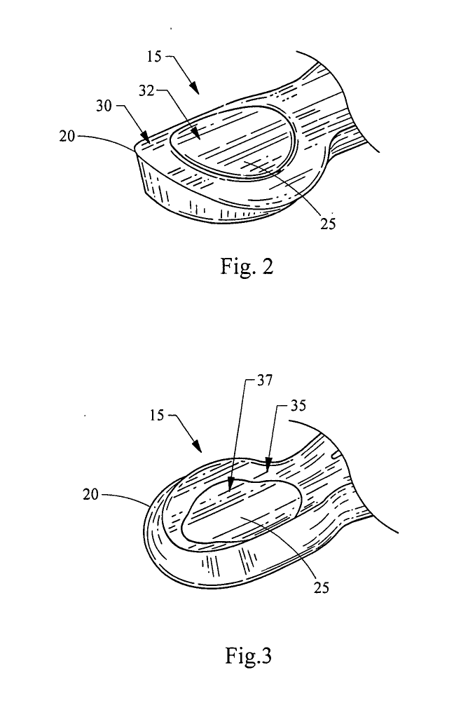

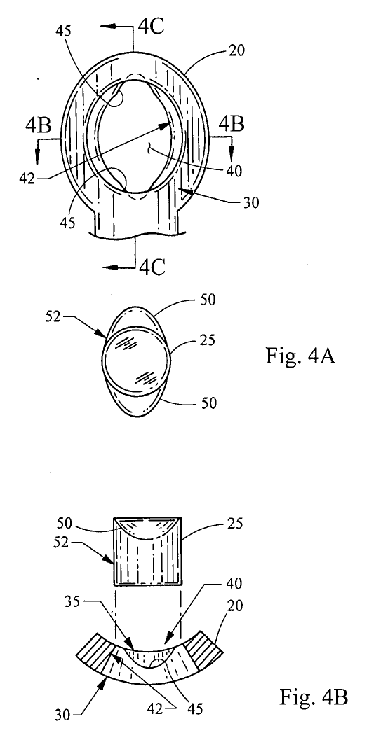

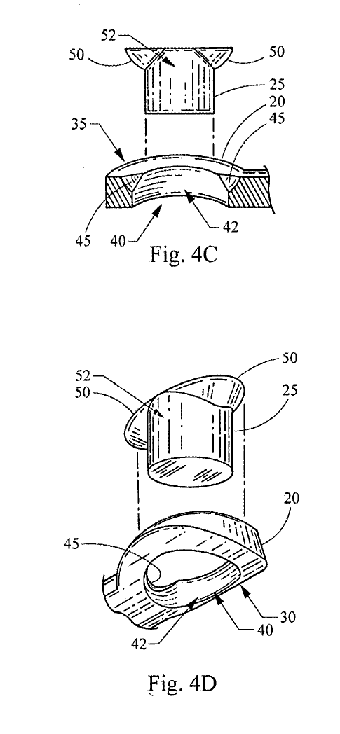

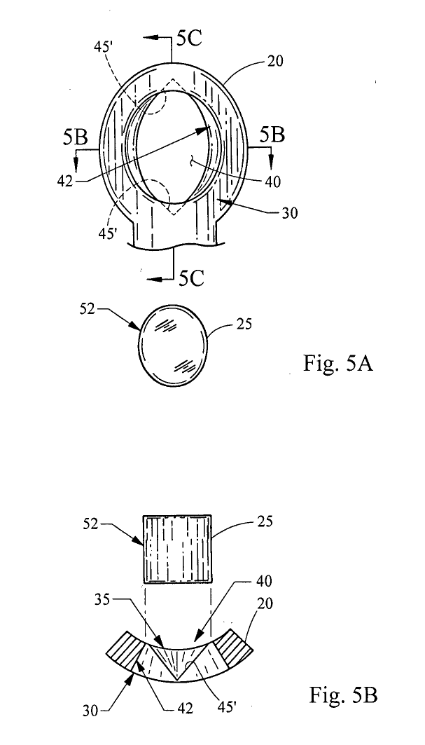

Each marker 15 includes an eyelet 20 having an opening 40 within which a radiopaque rivet 25 is disposed. The features of the opening 40, which is shown in FIGS. 4A and 5A according to two embodiments, will be described below. Referring to FIGS. 2 and 3, the eyelet 20 has an exterior surface 30 and an interior surface 35. The rivet 25 also has an exterior surface 32 and an interior surface 37. The interior surfaces 35, 37 face the lumen of the intraluminal medical device, i.e., the interior of the tubular structure 10, and the exterior surfaces 30, 32 face the vessel wall.

The exterior surface 32 of the radiopaque rivet 25 and the exterior 30 surface of the eyelet 20 may have an exterior radius of curvature which is substantially the same as the outer radius of the tubular structure 10 in a compressed state. The exterior radius of curvature may also be substantially the same as the outer radius of the tube from which the tubular structure 10 was formed, as will be described below.

Similarly, the interior surface 37 of the radiopaque rivet 25 and the interior surface 35 of the eyelet 20 may have an interior radius of curvature which is substantially the same as the inner radius of the tubular structure 10 in a compressed state. The interior radius of curvature may also be substantially the same as the inner radius of the tube from which the tubular structure 10 was formed, as will be described below.

Further, the exterior surface 32 of the radiopaque rivet 25 may be substantially flush with the exterior surface 30 of the eyelet 20. The interior surface 37 of the radiopaque rivet 25 may also be substantially flush with the interior surface 35 of the eyelet 20. Thus, the thickness of the radiopaque rivet 25 may be substantially the same as the thickness of the eyelet 20. The thickness of the radiopaque rivet 25 and the thickness of the eyelet 20 may also be substantially the same as the wall thickness of the intraluminal medical device, where the wall thickness is defined as the difference between the outer and inner radii of the tubular structure 10.

Preferably, the thickness of the radiopaque rivet 25, the thickness of the eyelet 20, and the wall thickness may be in the range of from about 0.1 mm to about 0.4 mm. According to one embodiment, the thicknesses may be in the range of from about 0.205 mm to about 0.260 mm. In another embodiment, the thicknesses may be in the range of from about 0.205 mm to about 0.245 mm. In another embodiment, the thicknesses may be in the range of from about 0.220 mm to about 0.260 mm.

According to one embodiment, the perimeter of the rivet 25 and the perimeter of the eyelet 20 may have a generally circular shape. The eyelet 20 may have an outer diameter in the range of from about 0.50 mm to about 0.75 mm. The outer diameter may also range from about 0.55 mm to about 0.70 mm. Alternatively, the outer diameter may range from about 0.62 mm to about 0.65 mm.

The perimeter of the radiopaque rivet 25 and the perimeter of the eyelet 20 may have other shapes, such as, for example, arcuate, oval, square, rectangular, diamond-like, triangular, or trapezoidal. The total area spanned by the exterior surface 30 of the eyelet 20 and the exterior surface 32 of the rivet 25 may range from about 0.18 mm² to about 0.45 mm². Alternatively, the total area may range from about 0.24 mm² to about 0.39 mm², or from about 0.30 mm² to about 0.33 mm².

FIGS. 4A-4D show different views of a radiopaque rivet 25 and an eyelet 20, according to one embodiment, prior to assembly and forming into a radiopaque marker 15. The eyelet 20 may be integrally formed as part of the intraluminal medical device, as will be discussed below. The radiopaque rivet 25 may be fabricated separately and then inserted and secured in the opening 40 of the eyelet 20 to form a radiopaque marker 15.

The opening 40 extends through the thickness of the eyelet 20 from one end, which includes one of the exterior surface 30 and the interior surface 35, to the other end, which includes the other one of the exterior surface 30 and the interior surface 35. The opening 40 is defined by at least an inclined surface 42 generally facing toward the one end of the eyelet 20 and a recessed region 45 generally facing toward the other end.

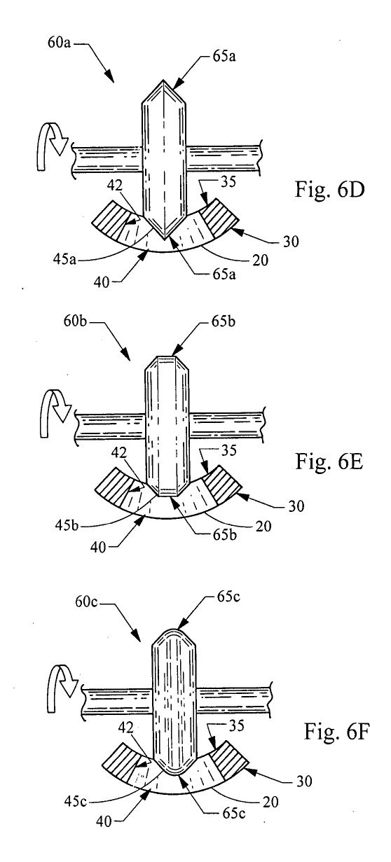

Referring to FIG. 4B, which shows a sectional view of the eyelet along section 4B-4B (demarcated in FIG. 4A), the opening 40 in the eyelet 20 may have a generally conical or tapered cross-section because of the inclined surface 42. A plane corresponding to section 4B-4B may lie perpendicular to the longitudinal axis of the tubular structure 10. At least a portion of the inclined surface 42 may extend entirely through the thickness of the opening 40, as shown in FIG. 4B. After insertion and securing of the radiopaque rivet 25 in the eyelet 20, the radiopaque rivet 25 also may have a generally conical or tapered cross-section, as shown in FIG. 8.

As mentioned above, in addition to the inclined surface 42, the opening 40 of the eyelet 20 may also include at least one recessed region 45. Preferably, the recessed region 45 is disposed about only a portion of the perimeter of the opening 40. Alternatively, the recessed region 45 may be disposed about the entire perimeter of the opening 40.

According to one embodiment, the recessed region 45 may extend only partially through the thickness of the eyelet 20 from one end, which includes one of the exterior surface 30 and the interior surface 35, to a position between the one end and the other end. The other end includes the other one of the exterior surface 30 and the interior surface 35. The exemplary recessed regions 45 shown in FIGS. 4A-4D extend approximately halfway into the thickness of the eyelet 20 from the interior surface 35 to a position between the interior surface 35 and the exterior surface 30. Alternatively, the recessed regions 45 may extend to other distances along the thickness of the eyelet 20, thereby having a smaller or larger size.

According to another embodiment, the recessed regions 45 may extend entirely through the thickness of the eyelet 20 from one end (including one of the exterior surface 30 and the interior surface 35) to the other end (including the other one of the exterior surface 30 and the interior surface 35 of the eyelet). For example, as shown in FIGS. 5A-5D, the recessed regions 45′ may extend entirely through the thickness of the eyelet 20 from the interior surface 35 to the exterior surface 30.

Preferably, the eyelet 20 may include an even number of recessed regions 45, such as, for example, two, four, six, or eight recessed regions 45, disposed about a portion of the perimeter of the opening 40. According to one embodiment, the eyelet 20 may include two recessed regions 45 positioned opposite of each other about the perimeter of the opening 40, as shown for example, in FIGS. 4A through 4D. The two recessed regions 45 may be positioned along a direction parallel to the line demarcating section 4C-4C in FIG. 4A. This direction may also be parallel to the longitudinal axis of the generally tubular structure 10.

According to one embodiment, the recessed regions 45 may have a curved cross-section when viewed along a first plane passing through the thickness of the eyelet 20 and through the centerline of the recessed regions 45, as shown, for example, in FIG. 4C, which corresponds to section 4C-4C of the eyelet 20 shown in FIG. 4A. The first plane may be parallel to the longitudinal axis of the tubular structure 10. Alternatively, the recessed regions 45′ may have a straight cross-section when viewed along the first plane, as shown for example in FIG. 5C, which corresponds to section 5C-5C of the eyelet 20 in FIG. 5A.

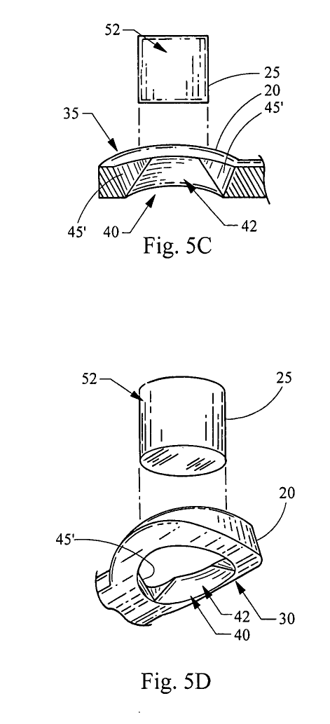

According to another embodiment, the recessed regions 45 may have a curved cross-section when viewed along a second plane passing through the thickness of the eyelet 20 and disposed perpendicular to the first plane, as shown, for example, in FIG. 4B. FIG. 4B corresponds to section 4B-4B of the eyelet 20 shown in FIG. 4A. The second plane may be perpendicular to the longitudinal axis of the tubular structure 10. According to alternative embodiments, however, the recessed regions 45 may have a triangular or a polygonal shape with straight and/or curved sides when viewed along the second plane. Examples of recessed regions (45a, 45b, and 45c) having such configurations are shown in FIGS. 6D-6F, which will be discussed below.

The radiopaque rivet 25 may have any shape suitable for insertion and securing within the opening of the eyelet. For example, one or more protrusions 50 may extend from the radiopaque rivet 25. Preferably, according to this embodiment, the radiopaque rivet 25 may include an even number of protrusions 50, such as two, four, six, or eight protrusions 50. The radiopaque rivet 25 may include two protrusions 50 positioned opposite of each other, for example, as shown in FIGS. 4A-4D. The two protrusions 50 may be positioned along a direction parallel to the longitudinal axis of the generally tubular structure 10. The protrusions 50 may extend, for example, from the interior surface 37 of the radiopaque rivet 25 and have a shape that generally corresponds inversely to the shape of the recessed regions 45. Alternatively, the radiopaque rivet 25 may not include protrusions 50. The radiopaque rivet 25 may have a more simple configuration, such as, for example, a cylindrical, disk-like, or rectangular shape. FIGS. 5A-5D show an exemplary radiopaque rivet 25 having a generally cylindrical shape.

After assembly and forming of the radiopaque marker 15, as will be described below, a first portion of the radiopaque rivet 25 engages the inclined surface 42 and a second portion of the radiopaque rivet 25 engages the recessed region 45 of the eyelet 20. For example, the first portion may include a portion of the outer surface 52 of the rivet 25, and protrusions 50 on the rivet 25 may constitute the second portion. Such protrusions 50 may be created on the rivet 25 during the forming of the radiopaque marker 15, or the protrusions 50 may be present on the radiopaque rivet 25 prior to the forming process and simply subject to further shaping during the forming of the marker 15. For example, the rivet 25 initially may not include protrusions 50 and have the generally cylindrical shape described above, or another shape. However, during the forming of the marker 15, one or more protrusions 50 may be created on the rivet 25 due to the presence of one or more recessed regions 45 in the eyelet 20.

Due to the engagement of respective first and second portions of the rivet 25 with the inclined surface 42 and the recessed region(s) 45 of the eyelet 20, the radiopaque rivet 25 may not be easily dislodged from the eyelet 20. According to one embodiment, protrusions 50 on the rivet 25 may engage with the recessed regions 45 to prevent the radiopaque rivet 25 from becoming dislodged in the direction of the vessel wall, and the outer surface 52 of the rivet 25 may engage with the inclined surface 42 to prevent the radiopaque rivet 25 from becoming dislodged from the eyelet 20 in the direction of the lumen. One possible benefit of a relatively large recessed region 45 that extends further into the thickness is greater structural integrity of the marker 15 after forming.

The eyelet 20 of the radiopaque marker 15 may be integrally formed with the generally tubular structure 10 of the intraluminal medical device. Thus, the eyelet 20 and the generally tubular structure 10 of the intraluminal medical device may be formed of the same material. Preferably, the eyelet 20 and the generally tubular structure 10 are formed of a biocompatible material, including, for example, at least one of: stainless steel, nickel, titanium, iron, cobalt, chromium, magnesium, aluminum, gold, silver, tantalum, palladium, platinum, iridium, niobium, tungsten, and alloys thereof; cellulose acetate, cellulose nitrate, silicone, cross-linked polyvinyl alcohol (PVA) hydrogel, polyurethane, polyamide, styrene isobutylene-styrene block copolymer, polyethylene teraphthalate, polyester, polyorthoester, polyanhydride, polyethersulfone, polycarbonate, polypropylene, high molecular weight polyethylene, polytetrafluoroethylene, or another biocompatible polymeric material, or mixtures or copolymers of these; polylactic acid, polyglycolic acid or copolymers thereof, a polyanhydride, polycaprolactone, polyhydroxybutyrate valerate or another biodegradeable polymer, or mixtures or copolymers of these; carbon or carbon fiber; ceramic materials, such as, for example, calcium phosphate; a protein, extracellular matrix component, coliagen, fibrin, or another biologic agent; or a suitable mixture of any of these.

Even more preferably, the generally tubular structure 10 and the eyelet 20 may be formed of a superelastic material. The term “superelastic material,” as used herein, refers to a material that exhibits a substantial amount of elastic (i.e., recoverable) deformation, or strain, in response to an applied stress. Typically, superelastic materials can achieve elastic strains of at least several percent. Upon removal of the applied stress, the elastic strain that was induced by the applied stress may be recovered and the material may return to its original, undeformed configuration. One example of a superelastic material is Nitinol, which is a superelastic nickel-titanium alloy that may achieve an elastic strain of about 8%. In contrast, conventional metal alloys, such as 304 stainless steel, typically achieve elastic strains of only a fraction of a percent. Materials exhibiting superelastic behavior also exhibit behavior that is referred to as shape memory or pseudoelastic. Superelastic materials are particularly advantageous for self-expandable stents. However, conventional materials may also be used.

The preferred superelastic material for the generally tubular structure 10 and eyelet 20 includes nickel and titanium. In one embodiment, the superelastic material is a binary nickel-titanium alloy, such as Nitinol. The nickel-titanium alloy may also include a ternary element, a quaternary element and/or additional elements.

The radiopaque rivet 25 of the radiopaque marker 15 may be fabricated from a radiopaque material. The term “radiopaque material,” as used herein, refers to a material that is substantially opaque to x-ray radiation and is thus readily visible using an x-ray imaging device, such as a fluoroscope. Preferably, the radiopaque material is also biocompatible. The radiopaque material may include, for example, gold, iridium, niobium, palladium, platinum, silver, tantalum, tungsten, or an alloy thereof. According to one embodiment, the radiopaque material may be gold. In another embodiment, the radiopaque material may be platinum. In another embodiment, the radiopaque material may be palladium.

The intraluminal medical device may be made from a thin-walled tube or sheet of any of the biocompatible materials described above. According to a preferred embodiment, a thin-walled tube made of a superelastic or shape memory material may be used. The superelastic material may be a binary nickel-titanium alloy, such as, for example, Nitinol. The nickel-titanium alloy may also include a ternary element, a quaternary element and/or additional elements. Thin-walled tubing made of superelastic nickel-titanium alloys is commercially available from companies such as, for example, Memry Corp. of Bethel, Conn. and Furukawa Techno Material Co., Ltd. of Kanegawa, Japan.

Next, at least one eyelet 20 and a generally tubular structure 10 may be formed from the thin-walled tube. The generally tubular structure 10 may include any desired pattern for the medical device. Conventional laser-cutting procedures known in the art may be employed to form the eyelet 20 and the tubular structure 10. Such procedures may involve, for example, loading a thin-walled tube into a laser cutting machine, such as those manufactured by, for example, Synova SA of Ecublens, Switzerland, and then cutting the tube using a laser under microprocessor control. The tube may be translated along and rotated about its longitudinal axis during the laser cutting procedure to form the eyelet 20, including the opening 40, and the pattern in the tube. Preferably, the laser is directed toward the longitudinal axis of the tube. After cutting, the resulting generally tubular structure 10 and eyelet 20 may be subjected to secondary processes such as being heat-treated and/or polished using methods known in the art.

The forming of the eyelet 20 includes forming an opening 40 extending through the thickness of the eyelet 30 from one end to the other end, where the one end includes one of the exterior surface 30 and the interior surface 35, and the other end includes the other one of the exterior surface 30 and the interior surface 35. The forming of the opening 40 includes at least forming an inclined surface 42 generally facing toward the one end and forming a recessed region 45 generally facing toward the other end of the eyelet 20. Conventional laser-cutting procedures known in the art, such as those described above, may be employed to form the inclined surface 42.

To form the recessed regions 45, one end of the eyelet 20, including one of the exterior surface 30 and the interior surface 35, may be ground or machined using a grinding tool or cutting tool of an appropriate shape. For example, as shown in FIGS. 6A-6F, the interior surface 35 of the eyelet 20 may be ground to form the recessed regions 45. The grinding tool or cutting tool may be formed of a hard material such as, for example, silicon carbide or aluminum oxide. Alternatively, the recessed regions 45 may be formed by laser cutting, as will be described below.

According to the embodiment of the method shown in FIGS. 6A-6F, a rotary grinding tool 60 may be employed. The diameter, thickness, and the shape of the edge(s) 65 of the grinding tool 60 may be selected based on the desired size and shape of the recessed regions 45. As shown in FIGS. 6A-6F, the grinding tool 60 may be a wheel. The rotary grinding tools 60a, 60b, and 60c shown in FIGS. 6D-6F have edges 65a, 65b, and 65c of different exemplary shapes.

Due to the curvature of the wheel, the recessed regions 45 may have a curved cross-section when viewed along a first plane passing through the thickness of the eyelet 20 and through the centerline of the recessed regions 45, as shown, for example, in FIG. 6C. This first plane may be parallel to the longitudinal axis of the tubular structure 10.

The recessed regions 45 may also have a curved cross-section when viewed along a second plane passing through the thickness of the eyelet and disposed perpendicular to the first plane, as shown, for example, in FIG. 4B. The second plane may be perpendicular to the longitudinal axis of the tubular structure 10. In another example, the recessed regions 45a may have the shape of a notch, as shown in FIG. 6D. Alternatively, the recessed regions 45b may have the concave three-sided shape shown in FIG. 6E. In yet another example, as shown in FIG. 6F, the recessed regions 45c may include both straight and curved portions. The shape of the recessed regions 45 formed may inversely correspond to the shape of the edges 65 of the grinding tool 60.

For example, the edges 65 of the grinding tool 60 may have a curved shape when viewed edge-on, as shown in FIG. 6B. Alternatively, as shown in FIG. 6D, the edges 65a of the grinding tool 60a may have a triangular shape with straight sides when viewed edge on. In another example, as shown in FIG. 6E, the edges 65b of the grinding tool 60b may have a polygonal shape with straight sides when viewed edge on. In yet another example shown in FIG. 6F, the edges 65c of the grinding tool 60c may include both straight and curved portions.

The recessed regions 45 shown in FIGS. 6B-6F extend approximately halfway into the thickness of the eyelet 20. Alternatively, the recessed regions 45 may be formed to have a smaller or larger size and thus may extend to any distance along the thickness of the eyelet 20. According to one embodiment, the recessed regions 45′ may extend substantially through the entire thickness of the eyelet 20, as shown for example in FIGS. 5B-5D. A possible benefit of a relatively large recessed region 45 is greater structural integrity of the marker.

Two recessed regions 45 positioned opposite of each other may be formed simultaneously using this embodiment of the method. Preferably, the two recessed regions 45 may be disposed along a line parallel to the longitudinal axis of the generally tubular structure 10.

According to another embodiment of the method, the recessed regions 45 may be formed in the eyelet 20 by laser cutting. Preferably, a multiple-axis (multi-axis) laser cutting apparatus may be used. With a multi-axis laser cutting apparatus, the laser may be directed toward an axis other than the longitudinal axis of the thin-walled tube. As a result, the angle of the cut with respect to the tube wall may be varied from the substantially perpendicular orientation attainable with traditional laser cutters. The recessed regions 45 may be formed in the eyelet 20 according to this embodiment of the method without grinding or machining.

In addition, the generally tubular structure 10, the eyelet 20, and the inclined surface 42 of the opening 40 may be formed using a multi-axis laser cutting apparatus, according to one embodiment of the method.

The radiopaque rivet 25 may be formed by conventional investment casting methods, or by other metal forming methods known in the art, such as, for example, stamping.

To form the radiopaque marker 15, the radiopaque rivet 25 may be inserted and secured within the opening of the eyelet 20. The securing of the radiopaque rivet 25 into the opening may include swaging, peening, upsetting, or flaring.

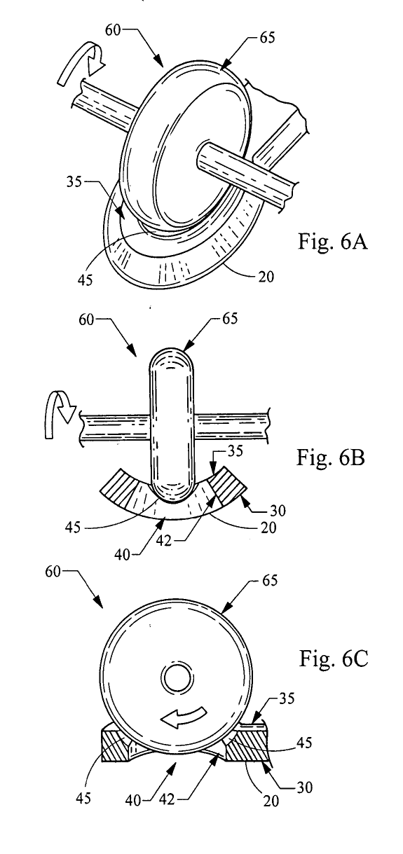

An embodiment of the method is shown in FIGS. 7A-7B. The radiopaque rivet 25 may be inserted into the eyelet 20, as shown in FIG. 7A, forming a rivet-eyelet assembly 55 (shown in FIG. 7B). The rivet-eyelet assembly 55 may be positioned on a stationary mandrel or lower die 70 having a radius of curvature which is substantially the same as that of the interior surface of the eyelet 20, as shown in FIG. 7B. Once in place, a movable upper die 75 having a radius of curvature which is substantially the same as that of the exterior surface 30 of the eyelet 20 may be lowered to contact and compress the radiopaque rivet 25 into the eyelet 20.

A force sufficient to deform the radiopaque rivet 25 to substantially match the size, shape, and curvature of the opening in the eyelet 20 may be applied. Standard metal forming presses known in the art with a dedicated upper die 75 and lower die 70 may be used.

FIGS. 8A-8B show another embodiment of the method. The radiopaque rivet 125 may be inserted into the eyelet 20 to form a rivet-eyelet assembly 55. The rivet 125 may not include protrusions 50 and may have a conical shape, as shown in FIG. 8A. The rivet-eyelet assembly 55 may be positioned on a stationary mandrel or lower die 75 that has a radius of curvature that is substantially the same as that of the exterior surface 30 of the eyelet 20, as shown in FIG. 8B. Once in place, a movable upper die 170 having a radius of curvature which is substantially the same as that of the interior surface 35 of the eyelet 20 may be lowered to contact and compress the rivet 125 into the eyelet 20. Alternatively, both the lower die 75 and the upper die 170 may move to compress the rivet 125 into the eyelet 20. The rivet 125 may be sized such that it protrudes from the eyelet 25 prior to the peening process.

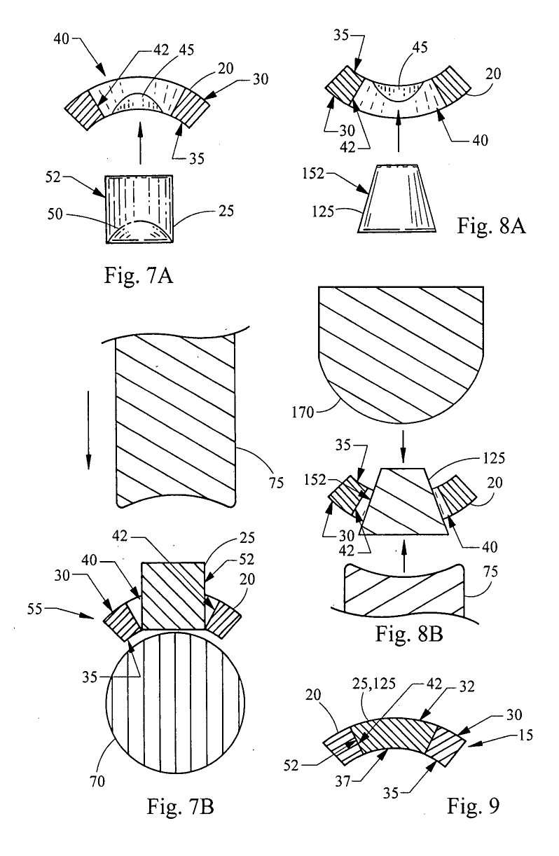

A sectional view of the radiopaque marker 15 after insertion and securing of the radiopaque rivet 25, 125 in the eyelet 20, according to one embodiment, is shown in FIG. 9.

After insertion and securing of the radiopaque rivet 25, 125 in the eyelet 20, a first portion of the radiopaque rivet 25, 125 engages the inclined surface 42 and a second portion of the radiopaque rivet 25, 125 engages the recessed region 45 of the opening 40 of the eyelet 20. The first portion may include a portion of the outer surface 52, 152 of the rivet 25. The second portion may include, according to one embodiment, one or more protrusions 50 of the rivet 25. For example, the second portion may include one, two, three, four, or more protrusions 50 to engage more than one recessed region 45 included in the opening 40 of the eyelet 20. According to one embodiment, two protrusions 50 on the rivet 25 may constitute the second portion. The protrusions 50 may be present on the radiopaque rivet 25 prior to the forming process and simply subject to further shaping during the forming of the marker 15. Alternatively, as in the process shown in FIGS. 8A-8B, protrusions may be created on the rivet 125 during the forming of the radiopaque marker 15.

Further, after insertion and securing of the radiopaque rivet 25, 125 in the eyelet 20, the exterior surface 32 of the radiopaque rivet 25, 125 may have an exterior radius of curvature which is substantially the same as the exterior radius of curvature of the exterior surface 30 of the eyelet 20. The exterior radius of curvature also may be substantially the same as the outer radius of the thin-walled tube from which the eyelet 20 was formed. Similarly, the interior surface 37 of the radiopaque rivet 25, 125 may have an interior radius of curvature which is substantially the same as the interior radius of curvature of the interior surface 35 of the eyelet 20. The interior radius of curvature also may be substantially the same as the inner radius of the thin-walled tube from which the eyelet 20 was formed.

Further, after insertion and securing of the radiopaque rivet 25 in the eyelet 20, the exterior surface 3.2 of the radiopaque rivet 25 may be substantially flush with the exterior surface 30 of the eyelet 20, and the interior surface 37 of the radiopaque rivet 25 may be substantially flush with the interior surface 35 of the eyelet 20. Thus, the thickness of the radiopaque rivet 25 may be substantially the same as the thickness of the eyelet 20. Also, the thickness of the radiopaque rivet 25 and the thickness of the eyelet 20 may be substantially the same as the wall thickness of the intraluminal medical device.

The intraluminal medical device may be any medical device suitable for implantation or insertion into the body, including, for example, a stent, stent graft, vascular graft, filter, embolization coil, catheter, or guide wire. Examples of stents that may be used in the present invention include self-expandable and balloon-expandable stents, including endovascular, biliary, tracheal, gastrointestinal, urethral, ureteral, esophageal and coronary vascular stents.

An intraluminal medical device including at least one radiopaque marker 15 has been described. The marker includes an eyelet 20 having an opening within which a radiopaque rivet 25, 125 is disposed. Preferably, the marker may not protrude from a surface of the intraluminal medical device. Thus, the profile of the medical device in a compressed or expanded state may not be substantially increased by the presence of the marker.

Furthermore, due to the engagement of a first portion of the radiopaque rivet 25, 125 with the inclined surface 42 of the eyelet 20 and the engagement of a second portion of the radiopaque rivet 25, 125 with the recessed region 45 of the eyelet 20, the marker may not be easily dislodged from the medical device.

Also, the marker may be manufactured by simple manufacturing processes.

Although the present invention has been described with reference to certain embodiments thereof, other embodiments are possible without departing from the present invention. The spirit and scope of the appended claims should not be limited, therefore, to the description of the preferred embodiments contained herein. All embodiments that come within the meaning of the claims, either literally or by equivalence, are intended to be embraced therein. Furthermore, the advantages described above are not necessarily the only advantages of the invention, and it is not necessarily expected that all of the described advantages will be achieved with every embodiment of the invention.

What is claimed is:

1. An intraluminal medical device comprising at least one radiopaque marker, the radiopaque marker comprising:
an eyelet comprising a thickness and an opening extending therethrough from a first side to a second side, the opening being defined by at least an inclined surface generally facing toward the first side and a recessed region generally facing toward the second side, the recessed region being disposed about only a portion of a perimeter of the opening; and
a radiopaque rivet disposed within the opening, the radiopaque rivet comprising a first portion engaging the inclined surface and a second portion engaging the recessed region.

2. The intraluminal medical device according to claim 1, wherein at least a portion of the first surface extends entirely through the thickness of the eyelet from the first side to the second side.

3. The intraluminal medical device according to claim 1, wherein the recessed region extends only partially through the thickness of the eyelet from the second side to a position between the second side and the first side.

4. The intraluminal medical device according to claim 1, wherein the recessed region extends entirely through the thickness of the eyelet from the second side to the first side.

5. The intraluminal medical device according to claim 1, comprising two recessed regions positioned opposite of each other about the perimeter of the opening.

6. The intraluminal medical device according to claim 1, wherein the recessed region comprises a curved cross-section.

7. The intraluminal medical device according to claim 6, wherein the recessed region comprises a curved cross-section along a first plane passing through the thickness of the eyelet and bisecting the recessed region.

8. The intraluminal medical device according to claim 7, wherein the recessed region comprises a curved cross-section along a second plane, the second plane passing through the thickness of the eyelet and disposed perpendicular to the first plane.

9. The intraluminal medical device according to claim 1, wherein the eyelet further comprises an exterior surface at one of the first side and the second side and an interior surface at the other of the first side and the second side, the exterior surface comprising a first radius of curvature and the interior surface comprising a second radius of curvature.

10. The intraluminal medical device according to claim 9, wherein the exterior surface is substantially flush with an exterior surface of the radiopaque rivet, and the interior surface is substantially flush with an interior surface of the radiopaque rivet.

11. The intraluminal medical device according to claim 10, further comprising a generally tubular structure, the radiopaque marker being disposed at an end of the generally tubular structure.

12. The intraluminal medical device according to claim 11, wherein the the first radius of curvature is substantially the same as an inner radius of the generally tubular structure in a compressed state, and the second radius of curvature is substantially the same as an outer radius of the generally tubular structure in a compressed state.

13. The intraluminal medical device according to claim 11, wherein the tubular structure and the eyelet are integrally formed.

14. An intraluminal medical device comprising at least one radiopaque marker, the radiopaque marker comprising:
an eyelet comprising a thickness and an opening extending therethrough from a first side to a second side, the opening being defined by at least an inclined surface generally facing toward the first side and two recessed regions disposed opposite of each other about a perimeter of the opening and generally facing toward the second side, wherein each recessed region is disposed about only a portion of the perimeter; and
a radiopaque rivet disposed within the opening, the radiopaque rivet comprising a first portion engaging the inclined surface and a second portion engaging the two recessed regions;
wherein the eyelet further comprises an exterior surface comprising a first radius of curvature and an interior surface comprising a second radius of curvature, the exterior surface being substantially flush with an exterior surface of the radiopaque rivet and the interior surface being substantially flush with an interior surface of the radiopaque rivet.

15. The intraluminal medical device according to claim 14, further comprising a generally tubular structure, the radiopaque marker being disposed at an end of the generally tubular structure,
wherein the first radius of curvature is substantially the same as an inner radius of the generally tubular structure in a compressed state, and the second radius of curvature is substantially the same as an outer radius of the generally tubular structure in a compressed state,
wherein the eyelet and the tubular structure are integrally formed, and wherein the intraluminal medical device is a stent.

16. A method of making an intraluminal medical device including at least one radiopaque marker, comprising:
providing a thin-walled tube;
forming a generally tubular structure and an eyelet from the thin-walled tube, the forming of the eyelet comprising forming an opening extending through a thickness of the eyelet from a first end to a second end, the forming of the opening comprising at least forming an inclined surface generally facing toward the first end and forming a recessed region generally facing toward the second end of the eyelet;
inserting a radiopaque rivet into the opening of the eyelet; and
securing the radiopaque rivet within the opening of the eyelet, a first portion of the radiopaque rivet engaging the inclined surface and a second portion of the radiopaque rivet engaging the recessed region.

17. The method of claim 16, wherein the forming of the generally tubular structure and the eyelet and the forming of the surface of the opening comprise laser cutting.

18. The method of claim 16, wherein the forming of the recessed region of the opening comprises grinding.

19. The method of claim 16, wherein the forming of the recessed region of the opening comprises laser cutting.

20. The method of claim 16, wherein the securing of the radiopaque rivet in the opening of the eyelet comprises swaging, the swaging comprising compressing and deforming the radiopaque rivet between a die and a mandrel.
